(19) United States
(12) Patent Application Publication
(10) Pub. No.: US 2026/0198887 A1
(43) Pub. Date: Jul. 16, 2026

(54) Wearable Medical Devices and Systems and Methods of Use Thereof

(71) Applicant: Anish Chogle, Irvine, CA (US)

(72) Inventor: Anish Chogle, Irvine, CA (US)

(21) Appl. No.: 19/448,502

(22) Filed: Jan. 14, 2026

Related U.S. Application Data
(60) Provisional application No. 63/745,133, filed on Jan. 14, 2025.

(51) Int. Cl. A61B 8/06; A61B 8/00; G06T 7/38; G06T 7/62

(52) U.S. Cl. CPC A61B 8/06; A61B 8/4227; A61B 8/466; A61B 8/5207; A61B 8/5223; G06T 7/38; G06T 7/62; A61B 2560/0487; G06T 2207/10004; G06T 2207/10136; G06T 2207/20081; G06T 2207/30104

(57) ABSTRACT

Systems for medical imaging enable real-time visualization of patient vasculature using augmented reality that includes a wearable device positioned on a patient's target body region and incorporates ultrasound transducers and Doppler sensors configured to acquire subsurface vascular structure data and blood flow characteristics. Sensor data is transmitted to a head-worn augmented reality visualization device used by a caregiver. The visualization device includes a camera configured to scan the target body region prior to placement of the wearable device, a display for presenting augmented reality content, and a processor configured to reconstruct a 3D representation of the anatomical surface from the camera data. The processor further fuses the subsurface vascular data with the reconstructed surface representation to generate a real-time, 3D vascular map. The vascular map is rendered as an augmented reality overlay aligned with the patient's body, thereby assisting caregivers with accurate visualization and guidance during vascular access procedures.

### CROSS-REFERENCE

The present application relies on U.S. Patent Provisional Application No. 63/745,133, titled “Wearable Medical Devices and Systems and Methods of Use Thereof” and filed on Jan. 14, 2025, for priority, which is herein incorporated by reference in its entirety.

### FIELD

The present specification relates to a wearable medical device for vascular access and vascular diagnosis. Specifically, the present specification provides a wearable imaging device for real time visualization and monitoring of vascular structures and blood flow within a patient's body.

### BACKGROUND

It is essential to be able to accurately and efficiently access veins and arteries for and during medical procedures such as IV catheterization, blood draw, and arterial line placement. Traditionally, these procedures rely on manual palpation or the use of handheld ultrasound devices, which require continuous manual operation and attention to external screens, often leading to multiple failed attempts. The current procedures for identifying and accessing veins or arteries are particularly problematic in patients with difficult venous access (DVA). The challenge is heightened in pediatric patients (whose veins and arteries tend to be smaller), elderly patients, and individuals in critical trauma situations where delays in access can have severe consequences.

Further, existing handheld ultrasound systems used for identifying the location of veins and arteries are generally bulky, require direct operator manipulation, and do not provide real-time feedback or hands-free visualization. In addition to ultrasound imaging devices for identification of the location of veins and arteries, Doppler ultrasound devices use sound waves to measure blood flow through blood vessels. However, ultrasound imaging and Doppler ultrasound devices are typically used separately. Therefore, there is a lack in the integration of ultrasound imaging devices with Doppler ultrasound for venous or arterial identification and blood flow measurement. Moreover, data derived from these devices are subject to manual interpretation which is prone to human errors.

Therefore, there is a need to integrate probing, visualization, and access devices, with intelligent analysis of the identified data to assist in both accurate visualization and making informed clinical and medical decisions, which can effectively reduce possibilities of human errors. There is also a need for hands-free, real-time guidance during medical procedures and continuous vascular health monitoring.

### SUMMARY

The following embodiments and aspects thereof are described and illustrated in conjunction with systems, tools and methods, which are meant to be exemplary and illustrative, and not limiting in scope. The present application discloses numerous embodiments.

The present specification discloses a system for medical imaging, comprising: a wearable device adapted to be positioned on a patient's target body region, the wearable device comprising: at least one ultrasound transducer configured to transmit acoustic signals into tissue and receive reflected echoes to generate first data indicative of subsurface vascular structures of the patient's target body region; at least one Doppler sensor configured to generate second data indicative of blood flow characteristics within blood vessels at the patient's target body region; a first communication unit in data communication with said at least one ultrasound transducer and said at least one Doppler sensor, wherein the first communication unit is configured to receive the first and second data for onward transmission; an augmented reality visualization device adapted to be head-worn by a caregiver, the augmented reality visualization device comprising: a screen configured to present augmented reality content within a field of view of the caregiver when worn; at least one camera configured to generate third data indicative of a scan of the patient's target body region prior to positioning of the wearable device on the target body region; a second communication unit configured to receive the first and second data from the first communication unit; a processor in data communication with said at least one camera, the second communication unit and at least one non-volatile memory for storing a plurality of programmatic instructions, which when executed, cause the processor to: receive first, second and third data; process the third data to reconstruct a three-dimensional representation of an anatomical surface of the patient's target body region; generate fused data by merging the first and third data, wherein the fused data is indicative of a real-time three-dimensional vascular map aligned with the three-dimensional representation of the anatomical surface; and render the three-dimensional vascular map as an augmented reality overlay that is displayed on the patient's target body region via the display.

Optionally, said aligning of the three-dimensional vascular map with the three-dimensional representation of the anatomical surface comprises spatially registering the first data with the third data using one or more coordinate transformation algorithms. Optionally, said aligning of the three-dimensional vascular map with the three-dimensional representation of the anatomical surface further comprises temporally synchronizing the first and third data based on corresponding acquisition timestamps.

Optionally, the three-dimensional vascular map is color-coded to represent at least one of vessel depth, vessel diameter, or the blood flow characteristics, wherein the blood flow characteristics include at least one of blood flow rate, blood flow direction, or blood flow velocity.

Optionally, the processor is further configured to execute one or more artificial intelligence algorithms to analyze the first, second and third data to identify and highlight one or more candidate veins or arteries suitable for vascular access based on at least vessel depth, vessel diameter, and blood flow characteristics, wherein said highlighting is enabled on the three-dimensional vascular map. Optionally, the processor is further configured to store the first, second and third data and to use the stored data as training data for subsequent execution of the one or more artificial intelligence algorithms.

Optionally, the augmented reality overlay is spatially locked to the patient's target body region such that the overlay remains aligned with the anatomical surface during movement of the caregiver's head.

Optionally, the processor is further configured to analyze the first and second data to monitor vascular health parameters including at least one of blood flow velocity, flow directionality, pulsatility index, resistive index, volumetric flow rate, vessel diameter, vessel cross-sectional area, vessel wall deformation, or changes in vessel compliance over time. Optionally, the processor is further configured to detect vascular abnormalities including at least one of abnormal blood flow, vessel constriction, vessel collapse, or clot formation based on the monitored vascular health parameters.

The present specification also discloses a method for medical imaging, comprising: positioning a wearable device on a patient's target body region; generating, using at least one ultrasound transducer, first data indicative of subsurface vascular structures of the patient's target body region; generating, using at least one Doppler sensor of the wearable device, second data indicative of blood flow characteristics within blood vessels at the patient's target body region; communicating the first and second data from the wearable device to an augmented reality visualization device worn on a head of a caregiver; acquiring, using at least one camera of the augmented reality visualization device, third data indicative of a scan of the patient's target body region prior to positioning of the wearable device on the target body region; processing the third data to reconstruct a three-dimensional representation of an anatomical surface of the patient's target body region; generating fused data by merging the first data and the third data, wherein the fused data is indicative of a real-time three-dimensional vascular map aligned with the three-dimensional representation of the anatomical surface; and rendering the three-dimensional vascular map as an augmented reality overlay that is displayed on the patient's target body region via a display of the augmented reality visualization device.

Optionally, aligning the three-dimensional vascular map with the three-dimensional representation of the anatomical surface comprises spatially registering the first data with the third data using one or more coordinate transformation algorithms. Optionally, aligning the three-dimensional vascular map with the three-dimensional representation of the anatomical surface further comprises temporally synchronizing the first data and the third data based on corresponding acquisition timestamps.

Optionally, rendering the three-dimensional vascular map comprises color-coding the vascular map to represent at least one of vessel depth, vessel diameter, or blood flow characteristics, wherein the blood flow characteristics include at least one of blood flow rate, blood flow direction, or blood flow velocity.

Optionally, the method further comprises executing one or more artificial intelligence algorithms to analyze the first, second, and third data to identify and highlight one or more candidate veins or arteries suitable for vascular access based on at least vessel depth, vessel diameter, and blood flow characteristics, wherein said highlighting is enabled on the three-dimensional vascular map. Optionally, the method further comprises storing the first, second, and third data and using the stored data as training data for subsequent execution of the one or more artificial intelligence algorithms.

Optionally, rendering the augmented reality overlay comprises spatially locking the overlay to the patient's target body region such that the overlay remains aligned with the anatomical surface during movement of the caregiver's head.

Optionally, the method further comprises analyzing the first and second data to monitor vascular health parameters including at least one of blood flow velocity, flow directionality, pulsatility index, resistive index, volumetric flow rate, vessel diameter, vessel cross-sectional area, vessel wall deformation, or changes in vessel compliance over time. Optionally, the method further comprises detecting vascular abnormalities including at least one of abnormal blood flow, vessel constriction, vessel collapse, or clot formation based on the monitored vascular health parameters.

The present specification also discloses a system for medical imaging, comprising: a wearable device adapted to be positioned on a patient's target body region, the wearable device comprising: at least one ultrasound transducer configured to transmit acoustic signals into tissue and receive reflected echoes to generate first data indicative of subsurface vascular structures of the patient's target body region; at least one Doppler sensor configured to generate second data indicative of blood flow characteristics within blood vessels at the patient's target body region; a first communication unit in data communication with said at least one ultrasound transducer and said at least one Doppler sensor, wherein the first communication unit is configured to receive the first and second data for onward transmission; an augmented reality visualization device adapted to be head-worn by a caregiver, the augmented reality visualization device comprising: a screen configured to present augmented reality content within a field of view of the caregiver when worn; at least one camera configured to generate third data indicative of a scan of the patient's target body region prior to positioning of the wearable device on the target body region; a second communication unit configured to receive the first and second data from the first communication unit; a processor in data communication with said at least one camera, the second communication unit and at least one non-volatile memory for storing a plurality of programmatic instructions, which when executed, cause the processor to: receive first, second and third data; process the third data to reconstruct a three-dimensional representation of an anatomical surface of the patient's target body region; generate fused data by merging the first and third data, wherein the fused data is indicative of a real-time three-dimensional vascular map aligned with the three-dimensional representation of the anatomical surface, wherein said aligning comprises spatially registering the first data with the third data using one or more coordinate transformation algorithms, and wherein said aligning further comprises temporally synchronizing the first and third data based on corresponding acquisition timestamps; render the three-dimensional vascular map as an augmented reality overlay that is displayed on the patient's target body region via the display.

Optionally, the processor is further configured to execute one or more artificial intelligence algorithms to analyze the first, second and third data to identify and highlight one or more candidate veins or arteries suitable for vascular access based on at least vessel depth, vessel diameter, and blood flow characteristics, wherein said highlighting is enabled on the three-dimensional vascular map. Optionally, the processor is further configured to store the first, second and third data and to use the stored data as training data for subsequent execution of the one or more artificial intelligence algorithms.

In embodiments, the present specification is directed towards a system for medical imaging, comprising: a biocompatible wearable device comprising: at least one doppler ultrasound transducer that generates a first signal indicative of imaging of a patient's vasculature; at least one doppler sensor that generates a second signal indicative of the patient's blood-flow velocity; a processor configured to utilize artificial intelligence to receive and analyze the first signal and the second signal; and a display in communication with the processor to receive the analyzed first signal and second signal, and display the analyzed first signal and second signal using augmented reality.

Optionally, the wearable device comprises a flexible sheet with a first outer surface and a second inner surface wherein the at least one ultrasound transducer and the at least one doppler ultrasound sensor are fixed to the second inner surface.

Optionally, the at least one ultrasound transducer and the at least one doppler ultrasound sensor form a network of sensors.

Optionally, the processor is embedded with the display.

Optionally, the processor is in communication with the biocompatible wearable device.

Optionally, the biocompatible wearable device further comprises at least one transmitter to communicate the first signal and the second signal to the processor.

Optionally, the display is a wearable display. Optionally, the display comprises wearable glasses. Optionally, the display is configured to project three-dimensional, color-coded maps of the patient's vasculature.

Optionally, the system further comprises at least one or more of: a temperature sensor, a heart rate monitor, and an SpO2 sensor. Optionally, the at least one or more of the temperature sensor, the heart rate monitor and the SpO2 sensor are integrated within the biocompatible wearable device. Optionally, the at least one or more of the temperature sensor, the heart rate monitor and the SpO2 sensor are in data communication with the processor.

Optionally, the processor further comprises a communication unit to wirelessly communicate with one or more computing devices.

Optionally, the biocompatible wearable device is reusable.

In some embodiments, the present specification is directed toward a method for medical imaging, comprising: applying a biocompatible wearable device embedded with sensors and a processor to receive and analyze data from the sensors, to a patient's body; receiving data from the sensors; analyzing received data from the sensors in real-time, wherein the analyzing comprises using artificial intelligence; and projecting analyzed data in real time through an augmented reality-based display.

Optionally, the sensors comprise a network of at least one ultrasound transducer and at least one doppler ultrasound sensor.

Optionally, the receiving the data comprises receiving images of the patient's veins and arteries. Optionally, the receiving the data comprises receiving the patient's blood-flow velocity data.

Optionally, the analyzing the received data comprises predicting medical conditions.

Optionally, the applying comprises wrapping the biocompatible wearable device around a limb or an abdomen of the patient.

Optionally, the method further comprises wirelessly communicating analyzed data for monitoring and displaying by a remote computing device.

The aforementioned and other embodiments of the present specification shall be described in greater depth in the drawings and detailed description provided below.

### BRIEF DESCRIPTION OF THE DRAWINGS

The accompanying drawings illustrate various embodiments of systems, methods, and aspects of the disclosure. Any person with ordinary skills in the art will appreciate that the illustrated element boundaries (e.g. boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1 is a block diagram of an exemplary system in accordance with the embodiments of the present specification;

FIG. 2A is a schematic drawing of an embodiment of wearable device which can be wrapped around a body surface of the patient, in accordance with the present specification;

FIG. 2B is a schematic drawing of a view of an inner surface of the device shown in FIG. 2A, in accordance with some embodiments of the present specification;

FIG. 2C is a schematic cross-section view along a portion of the embodiment of FIG. 2B, in accordance with the present specification;

FIG. 2D is a drawing of an exterior view of the embodiment of FIG. 2A when wrapped around a body surface of the patient, in accordance with some embodiments of the present specification;

FIG. 3 is a drawing showing an exemplary pair of glasses that can be used as an AR system, in accordance with the present specification; and

FIG. 4 is a flow chart showing an exemplary procedure of using the system shown in FIG. 1, in accordance with some embodiments of the present specification.

### DETAILED DESCRIPTION

The present specification is directed toward a wearable medical imaging device configured for real-time visualization and monitoring of vascular structures and blood flow. In one embodiment, the wearable device is wrapped around an arm or a limb of a patient. The wearable device includes embedded ultrasound transducers and Doppler sensors, which are configured to generate real-time imaging and blood-flow data. The device is configured to process the generated data using Artificial Intelligence (AI) algorithms for analysis of vein and artery structures, blood flow velocity, and for identification of optimal sites for procedures such as IV catheterization, blood draws, or arterial procedures. The analyzed data is projected onto a clinician's augmented reality (AR) glasses, displaying a color-coded vascular map of the patient's anatomy. Therefore, embodiments of the present specification use ultrasound technology, Doppler sensors, artificial intelligence (AI) for real-time data analysis, and augmented reality (AR) for hands-free display of vascular information. In some embodiments, systems and methods of the present specification supports various applications, including vascular access for intravenous (IV) catheterization, blood draws, arterial catheterization, chronic disease management, prenatal fetal monitoring, and the detection of vascular abnormalities such as deep vein thrombosis (DVT) and venous insufficiency, among several other possible medical uses. Additionally, embodiments of the present specification can be used to monitor vascular health over extended periods; and to generate alerts in cases of abnormal blood flow, vein collapse, or clot formation. Moreover, embodiments of the present specification can provide functionalities including prenatal fetal monitoring; providing real-time data on fetal blood flow and placental circulation during high-risk pregnancies.

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For the purpose of clarity, details relating to technical material that is known in the technical fields related to the invention have not been described in detail so as not to unnecessarily obscure the present invention.

In various embodiments, a computing device includes an input/output controller, at least one communications interface and system memory. The system memory includes at least one random access memory (RAM) and at least one read-only memory (ROM). These elements are in communication with a central processing unit (CPU) to enable operation of the computing device. In various embodiments, the computing device includes conventional computer components such as a processor, necessary non-transient memory or storage devices such as a RAM (Random Access Memory) and disk drives, monitor or display and one or more user input devices such as a keyboard and a mouse. In embodiments, the user input devices include an application that may be installed on the computing device and allows a user to select objects, icons, and text that appear on the display via a command or change parameters associated with various sensors that may be associated with a device. The computing device may also include software that enables wireless or wired communications over a network such as the HTTP, TCP/IP, and RTP/RTSP protocols. These elements are in communication with a central processing unit (CPU) to enable operation of the computing device. In various embodiments, the computing device may be a conventional standalone computer, a mobile phone, a tablet or a laptop. In some embodiments, the functions of the computing device may be distributed across multiple computer systems and architectures.

In some embodiments, execution of a plurality of sequences of programmatic instructions or code enable or cause the CPU of the computing device to perform various functions and processes. In alternate embodiments, hard-wired circuitry may be used in place of, or in combination with, software instructions for implementation of the processes of systems and methods described in this application. Thus, the systems and methods described are not limited to any specific combination of hardware and software.

The term “pulsatility index” used in this disclosure refers to a Doppler-derived metric that is used to quantify the variability of blood flow velocity within a blood vessel over a cardiac cycle, typically calculated as the difference between peak systolic velocity and end-diastolic velocity divided by the mean velocity. The pulsatility index is indicative of downstream vascular resistance and vessel elasticity.

The term “resistive index” used in this disclosure refers to a Doppler-derived parameter representing resistance to blood flow within a blood vessel, commonly calculated as the difference between peak systolic velocity and end-diastolic velocity divided by the peak systolic velocity. The resistive index is used to assess vascular impedance and can indicate conditions such as vessel narrowing or increased downstream resistance.

The term “vessel compliance” used in this disclosure refers to a mechanical property of a blood vessel that describes its ability to expand and contract in response to changes in internal blood pressure, typically expressed as the change in vessel diameter or volume per unit change in pressure. Reduced vessel compliance may indicate vessel stiffening, constriction, or pathological changes in the vessel wall.

The term “module”, “application” or “engine” used in this disclosure may refer to computer logic utilized to provide a desired functionality, service or operation by programming or controlling a general purpose processor. Stated differently, in some embodiments, a module, application or engine implements a plurality of instructions or programmatic code to cause a general purpose processor to perform one or more functions. In various embodiments, a module, application or engine can be implemented in hardware, firmware, software or any combination thereof. The module, application or engine may be interchangeably used with unit, logic, logical block, component, or circuit, for example. The module, application or engine may be the minimum unit, or part thereof, which performs one or more particular functions.

In the description and claims of the application, each of the words “comprise”, “include”, “have”, “contain”, and forms thereof, are not necessarily limited to members in a list with which the words may be associated. Thus, they are intended to be equivalent in meaning and be open-ended in that an item or items following any one of these words is not meant to be an exhaustive listing of such item or items, or meant to be limited to only the listed item or items. It should be noted herein that any feature or component described in association with a specific embodiment may be used and implemented with any other embodiment unless clearly indicated otherwise.

It must also be noted that as used herein and in the appended claims, the singular forms “a,” “an,” and “the” include plural references unless the context dictates otherwise. Although any systems and methods similar or equivalent to those described herein can be used in the practice or testing of embodiments of the present disclosure, the preferred, systems and methods are now described.

### System Overview

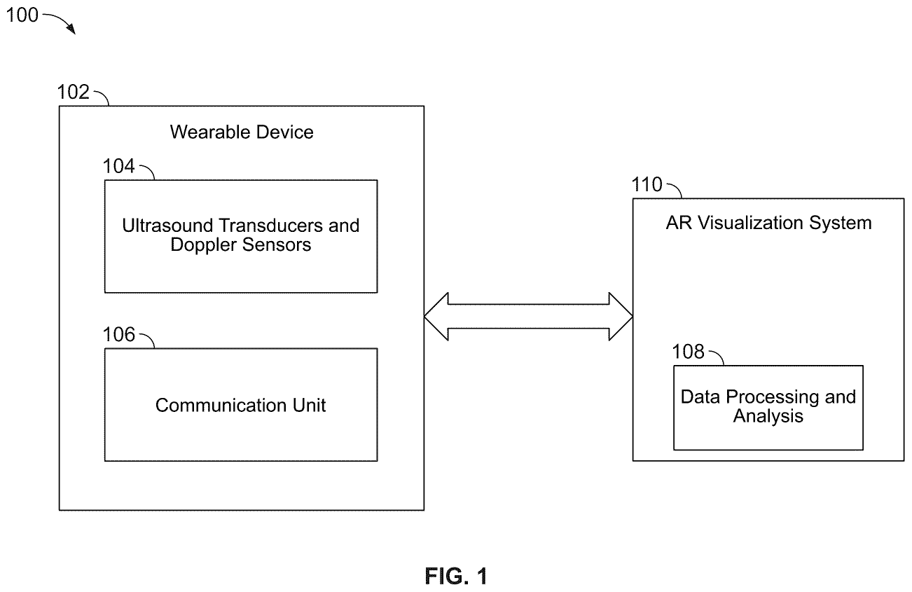

FIG. 1 illustrates an overview of an exemplary system 100 in accordance with the embodiments of the present specification. A wearable device 102 comprises sensors 104. Sensors 104 include at least one ultrasound transducer and/or at least one Doppler ultrasound sensor. In embodiments, multiple arrays of ultrasound transducers and Doppler ultrasound sensors are provided that form a dense network of sensors. Sensors 104 may additionally include other types of physiological sensors such as, and not limited to, temperature sensors, SpO2 sensors, and heart rate monitors. In embodiments, sensors 104 are embedded within wearable device 102. In one embodiment, device 102 is in the form of a flexible sheet, wherein sensors 104 are embedded on an inner surface of the sheet, where the inner surface is the surface that faces a body surface of a patient when device 102 is worn. An outer surface is the surface that is opposite to the inner surface.

In embodiments, a communication unit 106 is integrated into the wearable device 102 and is configured to transmit sensor data collected or received from the sensors 104 to a module 110, which in embodiments is an augmented reality (AR) visualization system. Similarly, the AR visualization system 110 includes a communication unit (not shown) integrated within the AR visualization system 110. The communication unit of the AR visualization system is configured to receive sensor data transmitted from the wearable device 102, and is in electrical and data communication with a data processing and analysis unit 108 also integrated within the AR visualization system 110. The communication unit of the AR visualization system is configured to communicate the sensor data received from the wearable device 102 to the data processing and analysis unit 108 and receive processed information from data processing and analysis unit 108. If required, the communication unit of the AR visualization system is also configured to transmit the processed information back to at least the wearable device 102, via a wireless communication link.

In embodiments, the wearable device 102 is controlled using an application, module or engine that may be installed on a mobile phone, tablet, laptop or any other computing device, wherein the computing device is in wired or wireless communication with the wearable device 102.

### Wearable Ultrasound Transducer and Doppler Sensor

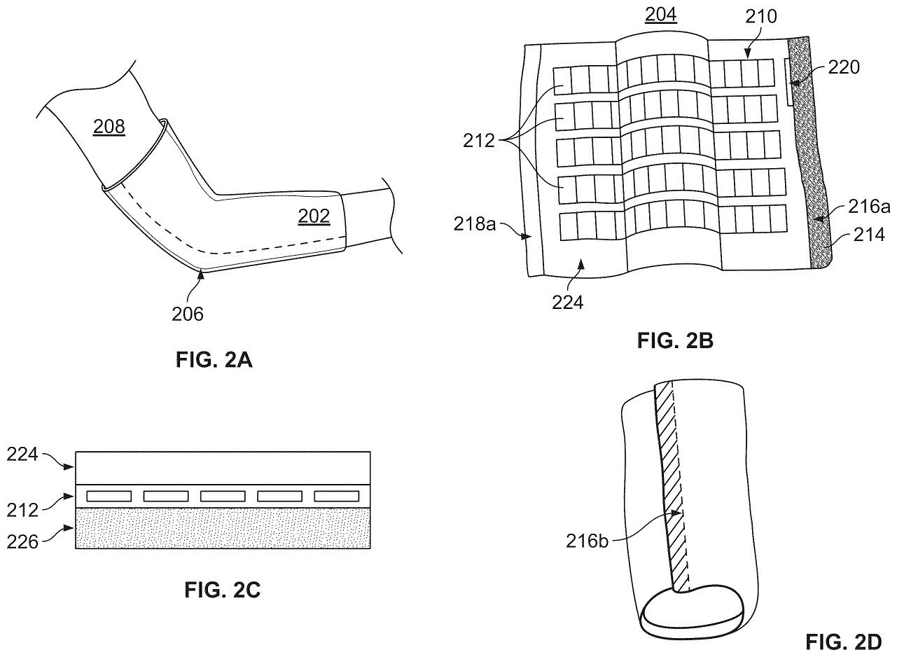

FIG. 2A illustrates wearable device 202 (shown as 102 in FIG. 1) which can be wrapped around a body surface of a patient, in accordance with the present specification. FIG. 2B is a schematic drawing of a view of an inner surface 204 of the device 202 shown in FIG. 2A, in accordance with some embodiments of the present specification. FIG. 2C is a schematic cross-section view along a portion of the device 202 of FIG. 2B, in accordance with the present specification. FIG. 2D is a drawing of an exterior view of the device 202 of FIG. 2A when wrapped around a body surface of the patient.

It should be noted herein that the device as described with respect to FIGS. 2A, 2B, 2C, and 2D is only exemplary. The device of the present specification may take any form factor, as long as it achieves the objectives of the present specification. For example, the form factor of the device may change depending upon the intended usage. In embodiments, a preferred attachment point/location is on an extremity (limb) of the human body in a use case contemplating IV insertion or blood draws, where the device form factor is such that it can easily be wrapped around a limb of a patient. In other embodiments, such as for monitoring of peripheral vascular disease, an exemplary placement site of the device is on the affected limb. In still other embodiments, for usage with fetal monitoring, an exemplary placement site is on the abdomen of the patient, and preferably over the location of the uterus. In still other embodiments, for usage with post-surgical monitoring or for monitoring of clots, an exemplary placement site is over the surgical site or where a clot has formed in the body. In embodiments, the device may include a cut-away, or elastic portion (with enhanced flexibility) for accommodating a movable joint of the patient (such as an elbow or knee) or a surgical wound or any other area which may affect positioning of the device.

The visible (outer) surface of device 202 may be smooth and includes embedded, flexible ultrasound patches that do not protrude from the inner surface. Further, device 202 includes Velcro strips on its exterior, allowing for secure attachment and adjustability around different extremities. Referring simultaneously to FIG. 1 and FIGS. 2A to 2D, device 202 is illustrated in a configuration that is suitable for wearing by wrapping around a limb of a patient. FIG. 2A shows device 202 wrapped around a forearm-elbow-bicep portion 206 of an arm 208 of a human being.

As shown in FIG. 2B, the interior of device 202 includes multiple arrays of sensors 210, where the sensors include ultrasound transducers and Doppler sensors, in embodiments. The ultrasound transducers are configured to transmit high-frequency acoustic signals into tissue and acquire reflected echoes, indicative of ultrasound data, from tissue interfaces and blood-filled vascular structures. The acquired ultrasound data is communicated to the AR visualization system 110 where the ultrasound data is processed by the data processing and analysis unit 108 which is configured to generate real-time images of the patient's vascular structure or anatomy and enable assessment of vascular parameters such as, for example, vessel diameter, vessel cross-sectional area, vessel wall deformation, and changes in vessel compliance over time.

The Doppler sensors provide real-time data on blood flow characteristics (such as, for example, directionality, velocity, pulsatility index, resistive index, and volumetric flow rate), aiding in the detection of vascular abnormalities such as blood clots and vein occlusions. In embodiments, arrays of sensors 210 are aligned in a plurality of parallel rows 212 in order to cover a large portion of inner surface 204, while providing space for margins along all four edges of the device 202, which, in an embodiment is rectangular. In embodiments, device 202 may include a cut-out portion for accommodating a joint (such as an elbow) of the patient and allowing for the device to be positioned so that it rests flat on the skin surface of the patient.

In various embodiments, rectangular device 202 has a length ranging from 18 to 40 centimeters (cm) and a width ranging from 8 to 10 cm. In one embodiment, the length of device 202 is 30 cm and the width is 12 cm. In some embodiments, each row 212 has a length ranging from 16 to 38 cm and a width in a range of 1.5 to 3.5 cm.

In one embodiment, where device 202 has a length of 30 cm and width of 12 cm, each row 212 has a length of 28 cm and a width of 2.5 cm. In the same embodiment, a space of 0.5 to 1.5 cm between each adjacent parallel row 212 is provided and a margin of at least 1 to 2 cm along the edges is available.

An attachment system 214 is provided on at least one of the edges of device 202. In one embodiment, a peel and stick (Velcro) system is used as attachment system 214. Attachment system 214 allows for secure attachment and adjustability of wearable device 202 around different extremities. In this case, at least one first interior edge 216a (with a first exterior edge 216b opposite to the at least one first interior edge 216a) of device 202 is fitted with a first surface of the peel and stick system. At least one second exterior edge (not shown, with a second interior edge 218a opposite to the at least one second exterior edge) of device 202, parallel to first interior edge 216a and first exterior edge 216b, is fitted with a complementary surface of the peel and stick system.

Wearable device 202 is primarily made from a flexible, biocompatible material, allowing the device to conform to a variety of anatomical regions including the arms, legs, neck, and the abdominal area. In some embodiments, an exterior layer 224 of the inner surface 204 of device 202 is made using a flexible material such as for example thermoplastic polyurethane (TPU). In embodiments, exterior layer 224 covers and protects arrays of sensors 210 embedded within device 202. Referring to FIG. 2C, an interior surface or layer 226 of device 202 is fabricated from a hydrogel, upon which the arrays of sensors 210 rest. Thus, the inner surface 204 that faces the patient's body is a composite made from the interior surface or layer 226 of hydrogel and the exterior layer 224 is formed from TPU, wherein the arrays of sensors 210 are sandwiched between the interior layer 204 and exterior layer 224. It should be noted herein that each of the inner layer 204 and outer layer 224 can be made from any biocompatible material such that the objectives of the present specification are achieved.

Referring back to FIG. 1, a communication unit 106 is embedded within wearable device 102. Communication unit 106 is preferably a wireless transmitting (or transmitting and receiving) unit that enables at least the communication of the sensor data collected by sensors within arrays 210 to the corresponding communication unit of the AR visualization system 110. Therefore, communication unit 106 is in electrical and data communication with arrays of sensors 210 shown in FIG. 2B. FIG. 2B illustrates a Bluetooth transmitter unit 220 embedded on inner surface 204 of wearable device 202, which, in some embodiments, is used and configured to function as communication unit 106. Transmitter unit 220 is positioned, in one embodiment, along an inner edge of attachment system 214.

In some embodiments, disposable sterile covers are used to encase wearable device 202 prior to each patient use. These covers are fabricated from acoustically transparent and biocompatible materials, such as medical-grade polyurethane films, thermoplastic polyolefins, or silicone sheeting. The selected material enables clear transmission of ultrasound and Doppler signals without distortion and does not interfere with any AR-based optical or wireless communication functionality. The covers maintain sterility while preserving signal integrity and imaging quality during vascular mapping and hemodynamic assessments.

### AR Visualization System

In embodiments, as shown in FIG. 1, the sensor data received from wearable device 102 is used by an augmented reality (AR) visualization system 110 which is configured to process, fuse, and render the sensor data as an augmented reality overlay incorporating additional analytical information. AR system 110 typically comprises a headset with a processing system, memory, and display integrated into the physical headset such that, when a user wears the headset, his or her entire field of vision is occupied by the display, which may present to the user any type of graphic, text, video, and/or other images. AR system 110 comprises a data processing and analysis unit 108 that is configured to process the sensor data received from wearable device 102, an optical viewing unit or a camera that is configured to scan the target body surface of a patient where device 102 is positioned, and a projector or display/screen that is configured to render a 3D representation of the body surface, the 3D representation being reconstructed by the data processing and analysis unit 108 by combining the sensor data associated with at least the ultrasound and Doppler sensors and optical data associated with the optical viewing unit. In embodiments, the reconstructed three-dimensional representation is rendered and presented by AR visualization system 110 and appears, to a user of the AR visualization system, superimposed onto the surface of the body of the patient at a location corresponding to where the wearable device 102 was previously positioned (since the wearable device 102 is removed prior to rendering the 3D representation on the patient's body).

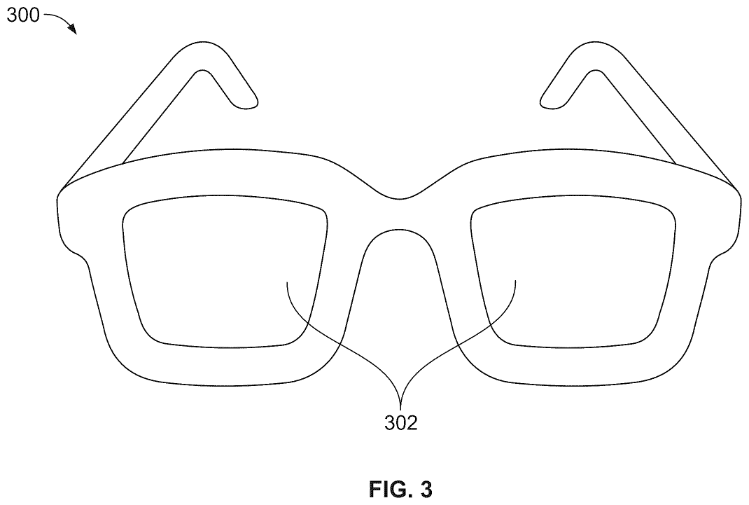

FIG. 3 illustrates an exemplary pair of glasses 300 that can be used as AR visualization system 110, in accordance with the present specification. AR visualization system 110 is configured to display a color-coded vascular map, generated using the sensor and optical data processed by unit 108, on projector or display/screen 302 of the glasses 300 to assist physicians with hands-free guidance to navigate veins accurately. In embodiments, the color coding (also referred to as ‘clinical color coding’) of the vascular map is based on real-time analysis of blood flow velocity, flow directionality, vessel diameter, and compliance-related indices derived from Doppler and ultrasound data. Venal structure or subsurface data is provided by ultrasound transducers and flow data is provided by Doppler sensors. Processing unit 108 is configured to analyze the vein size/structure and flow data. Subsequently, AR visualization system 110 generates the vascular map in real-time to assist navigation.

In embodiments, based on continuous or periodic analysis of ultrasound-derived structural or subsurface data and Doppler-derived blood flow data, the system supports detection of conditions indicative of vascular abnormalities, including but not limited to abnormal blood flow, vessel constriction, vessel collapse, and potential clot formation. Thus, in addition to generating the real-time vascular map, the AR visualization system 110, in association with the data processing and analysis unit 108, is configured to monitor vascular health parameters over extended periods of time. In some embodiments, one or more predefined thresholds corresponding to the monitored vascular parameters are programmed into the wearable device 102 of the present specification. Such parameters may include, for example, blood flow velocity, flow directionality, pulsatility or resistive indices, volumetric flow rate, vessel diameter, and vessel cross-sectional area. In embodiments in which the wearable device 102 includes higher-resolution ultrasound sensing capabilities, additional parameters such as vessel wall deformation, and/or changes in vessel compliance over time may also be derived and monitored.

In embodiments, the predetermined thresholds are programmed via a software application module or engine executable on a computing device (such as, for example, a smart phone, a laptop or a tablet) that is associated with and in data communication with the wearable device 102. In embodiments, the predetermined thresholds are programmed into the wearable device 102 and may be customized on a per-patient basis to account for individual physiological baselines or clinical requirements. When one or more of these parameters deviate from a corresponding predetermined threshold or range, the system generates an alert indicative of a potential vascular condition requiring attention. In embodiments, the alerts are communicated to the patient and/or a caregiver or clinician. In some embodiments, patient alerts are generated directly on the wearable device 102 and may include auditory notifications, tactile feedback such as vibration, or combinations thereof, thereby enabling timely awareness. In embodiments, alerts are communicated to caregivers or clinicians through the application installed on a computing device (such as, for example, a mobile phone) and linked (via Bluetooth or other communications protocol) with the wearable device 102. Once a parameter exceeds a corresponding predetermined threshold or range, the wearable device 102 transmits the data and corresponding alert to the application (on the computing device) in real time. From the computing device, notifications can be sent to a clinician dashboard, a secure messaging system, or an electronic health record interface, depending on the system in use.

In embodiments, during a treatment or a diagnostic procedure, a clinician wears AR system 110 initially to scan an extremity of the patient's limb where wearable device 102 will be positioned. AR system 110 includes at least one embedded optical viewing unit, such as a camera, and AR projection capabilities. The optical viewing unit is configured to scan the extremity of the patient (prior to positioning the wearing device 102 on the patient) while the projector or display/screen renders a 3D image or representation of the scanned extremity. The optical viewing unit captures raw optical or visual data, which may be in the form of RGB-D (Red, Green, Blue, and Depth) data or 3D point cloud data (x, y, z axes). The raw visual data format includes color information and depth information structured in arrays or matrices that represent spatial coordinates of the scanned surface in three-dimensional space. It should be appreciated that the color information refers to raw optical RGB data used for surface geometry and spatial reconstruction of the patient's limb. However, clinical color-coding displayed to the clinician is applied after data fusion and is derived from processed ultrasound and Doppler parameters (for example, flow velocity, vessel size, and compliance), not from raw optical color values. The data processing and analysis unit 108 additionally merges the raw visual data from the optical viewing unit, with the sensor data stream received from the ultrasound and Doppler sensors embedded in wearable device 102. Sensor and optical data from the two sources (the ultrasound and Doppler sensors as well as the optical viewing unit) is combined or merged so that spatial and temporal information from the two sources is aligned and overlaid.

Specifically, optical or visual data acquired by the optical viewing unit is processed by the data processing and analysis unit 108 to reconstruct a 3D surface model of the limb on which the wearable device 102 is positioned. Subsurface vascular structures detected by the ultrasound sensors are spatially registered (by the data processing and analysis unit 108) to the reconstructed 3D surface model such that the ultrasound sensor data or subsurface data (generated by the ultrasound sensors and indicative of the subsurface vascular structures) is mapped onto a common coordinate frame with the 3D surface model. This spatial registration is performed using coordinate transformation algorithms executed by the data processing and analysis unit 108 of the AR system 110. Persons of ordinary skill in the art would appreciate that the coordinate transformation algorithms refer to computational methods that convert spatial data from one coordinate system into another, enabling data acquired from different sensors to be expressed within a common reference frame.

Following spatial registration, the optical or visual data and ultrasound sensor data are temporally synchronized (by the data processing and analysis unit 108) based on corresponding acquisition timestamps or frame alignment to ensure that the displayed subsurface vascular structures correspond accurately with the real-time position and movement of the limb surface. Additionally, the Doppler sensor data is also temporally synchronized with the ultrasound sensor data and the optical or visual data. The spatially aligned and temporally synchronized datasets are then fused (by the data processing and analysis unit 108) to generate a color-coded three-dimensional vascular map aligned with the 3D surface model. This fused representation simultaneously depicts surface-level anatomical features derived from the optical data and subsurface vascular structures derived from the ultrasound data, thereby providing the clinician with a comprehensive and real-time visualization of vascular anatomy in the context of the surface-level anatomical features. In embodiments, the color-coded vascular map is derived by mapping flow-related parameters (such as velocity, direction, and vessel size) to distinct visual overlays, with color schemes either following clinical conventions (e.g., red for arterial, blue for venous) or customizable based on user preference. These colors are rendered on top of the 3D surface model using real-time, synchronized data from optical, ultrasound, and Doppler sensors, ensuring accurate visual guidance.

### Data Processing and Analysis

In some embodiments, each patient monitoring device, sensor, and/or processing system, including the data processing and analysis unit 108, is configured to execute programmatic instructions. The AR visualization system 110 includes at least one wired and/or wireless communication interface comprising a receiver and/or transmitter capable of transmitting and receiving data. The data processing and analysis unit 108 further comprises at least one processor configured to execute programmatic instructions, memory configured to store programmatic instructions and related data, and software module or engine comprising a plurality of programmatic instructions that, when executed by the at least one processor, cause performance of the processes and functionalities described herein.

AR visualization system 110 includes the data processing and analysis unit 108 (computing unit) comprising a processor and memory. The communication unit 106, in embodiments, is in wireless communication with a corresponding communication unit of the AR visualization system 110. The communication unit of the AR visualization system 110 is in data communication with the data processing and analysis unit 108. This enables sensor data, received by the communication unit of the AR visualization system 110 from the communication unit 106, to be communicated to the data processing and analysis unit 108 for processing. Thus, the data processing and analysis unit 108 is configured to communicate with the communication unit 106 on the wearable device 102 via a communication unit associated with AR visualization system 110, to receive the sensor data from at least the ultrasound sensors and/or the Doppler sensors and process the sensor data in combination with optical or visual data from the optical viewing unit or camera positioned on the AR visualization system 110. In alternate embodiments, the data processing and analysis unit 108 is configured to communicate directly with the communication unit 106 on the wearable device 102 to receive the sensor data from at least the ultrasound sensors and/or the Doppler sensors and process the sensor data in combination with optical or visual data from the optical viewing unit or camera positioned on the AR visualization system 110.

In some embodiments, data processing and analysis unit 108 is configured to execute one or more programmable instructions to process and/or analyze at least the sensor data from ultrasound transducers and Doppler sensors in combination with optical or visual data from the optical viewing unit, in real time. In embodiments, data processing and analysis unit 108 is configured to continuously analyze vascular structures using the sensor data. Unit 108 is further configured to process the received sensor data to extract spatial and physiological characteristics of vascular anatomy, including spatial dimensions, depth, orientation, and vessel diameter as well as blood flow characteristics. Based on this analysis, the unit 108 is configured to determine the structure, location, and dimensions of veins and arteries and evaluates the suitability of individual vessels for vascular access. Thus, unit 108 is configured to: identify optimal veins or arteries for venous or arterial access based on factors such as, but not limited to, vein depth, size, and blood flow velocity; monitor for abnormal blood flow patterns, such as reduced flow indicative of deep vein thrombosis (DVT), venous collapse, or vessel stenosis; provide adaptive real-time alerts and feedback, adjusting recommendations based on the patient's dynamic conditions (including and not limited to: dehydration, movement, or fluctuations in blood pressure).

Furthermore, the data processing and analysis unit 108 is configured to employ one or more AI algorithms, including machine-learning algorithms, that are trainable using data derived from prior procedures in order to improve predictive accuracy and vascular selection performance over time, thereby optimizing procedural outcomes. In some embodiments, the AI algorithms comprise one or more deep learning models trained using supervised learning techniques on a large dataset that includes labeled examples of vascular structures, blood flow characteristics, and corresponding surface anatomy maps. The training dataset supports labeling of individual vascular features with attributes including, but not limited to, vessel type (vein or artery), depth, diameter, blood flow rate or velocity, and an indication of suitability for vascular access.

During training and validation, model outputs are evaluated against ground-truth labels to identify misclassification of vascular types or inaccurate vascular access recommendations, and model parameters are iteratively adjusted to reduce such errors. In some embodiments, the configuration of the data processing and analysis unit 108 includes assigning relative weights to different input parameters when generating accessibility or suitability scores for candidate vessels. For example, input parameters such as blood flow characteristics and vessel diameter may be weighted more heavily in determining vascular accessibility, while spatial alignment accuracy between surface and subsurface features is also weighted to ensure reliable visualization and targeting. An input parameter such as vessel depth may additionally be considered in the weighting scheme, but with lower relative priority compared to input parameters such as flow and diameter, thereby reflecting clinical relevance while maintaining robust decision-making.

In an exemplary use-case scenario of a pediatric patient with dehydration and difficult venous access, the system of the present specification prioritizes deeper veins with adequate flow over collapsed or superficial vessels. Vessel depth and blood flow velocity are weighted most heavily to identify stable targets. Vessel diameter is also considered, but it is balanced against depth to avoid non-viable options. Flow direction and pulsatility are primarily used to differentiate veins from arteries, not to drive access decisions. The AR display highlights optimal veins in bright, color-coded overlays while de-emphasizing low-perfusion or deeply buried vessels. In this scenario, the system makes it much easier for clinicians to identify the most suitable access site in real time.

The data processing and analysis unit 108 is further configured to provide adaptive, real-time feedback, wherein vascular access recommendations are dynamically adjusted based on changing physiological and contextual conditions of the patient. Such conditions may include, but are not limited to, the patient's level of hydration, body posture, type and extent of movement, and fluctuations in blood pressure and/or SpO₂. In some embodiments, unit 108 receives sensor data from one or more additional sensors configured to monitor the patient wearing the wearable device 102, or from one or more remote patient monitoring devices that acquire and analyze patient data from sensors other than the ultrasound and Doppler sensors described herein. In embodiments, such additional sensors are in data communication with unit 108 and/or the wearable device 102 via wired or wireless communication links, including Bluetooth®, Wi-Fi, or cellular transmission, thereby enabling real-time integration of multi-parameter patient data into the recommendation and feedback process.

By way of example, hydration status may be monitored using bioimpedance sensors configured to estimate body fluid distribution. Patient posture and orientation may be monitored using inertial measurement units comprising accelerometers, gyroscopes, and, in some embodiments, magnetometers or tilt sensors. The status and type of patient movement may be monitored using motion sensors such as accelerometers and gyroscopes. Fluctuations in blood pressure and related hemodynamic parameters may be monitored using cuff-based or cuffless blood pressure sensors, and photoplethysmography sensors for monitoring SpO₂ and heart rate.

In some embodiments, sensor data from the ultrasound sensors, embedded in wearable device 102, is in a DICOM (Digital Imaging and Communications in Medicine) format. This data consists of grayscale images that represent cross-sectional views of the subsurface structures. The sensor data also includes Doppler data that is indicative of blood flow rate or velocity. Doppler data is structured in matrices where pixel intensity represents tissue density or flow rate.

### Communications Unit

Wearable device 102 includes the communication unit 106 that is configured to receive at least first data and second data (collectively referred to as ‘sensor data’) from the ultrasound transducers and Doppler sensors 104, respectively, and transmit the sensor data to the AR visualization system 110. Further, AR visualization system 110 comprises a corresponding communication unit that is configured to receive the sensor data from unit 106, and any other sensor(s) and/or remote patient monitoring device(s) or any other computing device using Bluetooth, WIFI, or cellular transmission. Since the communication unit of the system 110 is in data communication with the data processing and analysis unit 108, the communication unit communicates the received sensor data (from unit 106) to the data processing and analysis unit 108. The communication unit of system 110 is also configured to wirelessly transmit data processed by unit 108 back to the communication unit 106.

In embodiments, each of the communication unit 106 and the corresponding communication unit of system 110 is embedded with wireless receiving and/or transmitting circuits to enable wireless communication. Wireless communication methods used herein may include Bluetooth, Bluetooth Low Energy (BLE), Long Term Evolution (LTE), Near-Field Communication (NFC) or Wi-Fi. Therefore, wearable device 102 and AR system 110, are configured to wirelessly communicate with each other and potentially with additional health-related sensors, personal health monitors, patient monitors, remote servers, clinical monitoring and diagnostic devices, databases (Electronic Health Records (EHR)), or other types of computing devices such as mobile phones. Thus, in embodiments, system 100 of the present specification ensures that clinicians, physicians, and any other personnel associated with the patient have real-time access to patient data, even remotely.

### AR Visualization Process

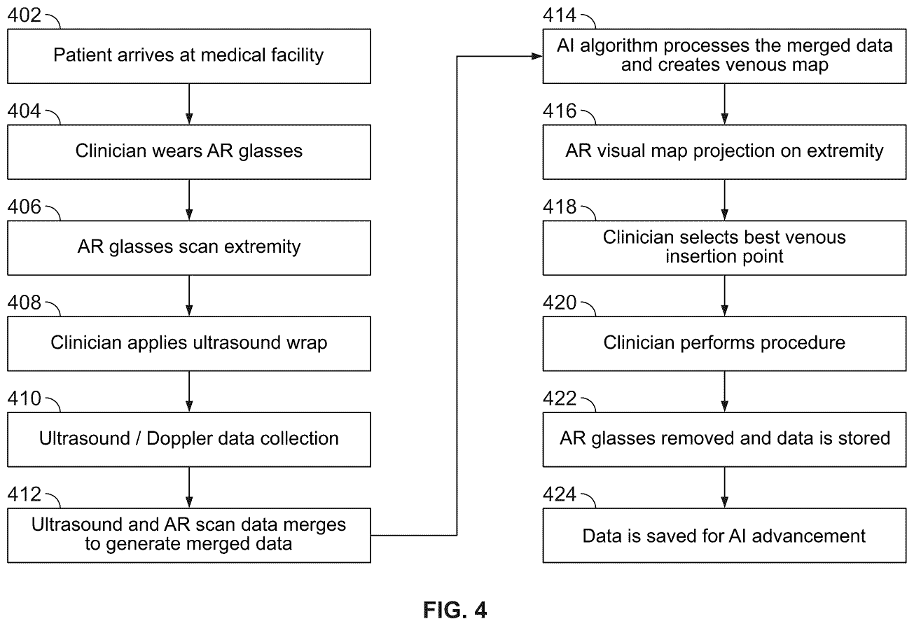

FIG. 4 is a flow chart describing an exemplary procedure for using system 100 of FIG. 1, in accordance with some embodiments of the present specification. At step 402, a patient arrives at a medical facility for a clinical procedure requiring, for example, vascular access such as for blood collection, or for placement of an intravenous (IV) needle for treatment or for diagnostics. Alternatively, a clinician visits a patient for one of the purposes mentioned previously.

At step 404, the clinician prepares and wears AR visualization system 110, which herein for exemplary purposes includes a headset such as goggles or glasses. If required, the clinician enables or activates the operation of the AR glasses. In embodiments, the AR visualization system 110 is activated by powering on using a switch. Thereafter, the AR visualization system 110 may be paired to the wearable device 102 using a Bluetooth or other short range pairing protocol. In embodiments, a device ID may need to be associated with the AR visualization system 110. The AR glasses comprise a camera and a projector or display/screen, in addition to a processing or computing unit or chip.

At step 406, once the AR glasses are activated, the clinician, while wearing the glasses, focuses the embedded camera in the AR glasses towards the patient's target site (where the target site is selected by the clinician) for drawing blood, or for any other purposes within the scope of applications of the present specification. The embedded camera, or multiple cameras, in the AR glasses scan the target body surface to generate optical or visual data that is processed to generate a 3D reconstruction of the target site. The target site is also the site where a wrap embodiment of the wearable device (device 102) is subsequently worn by the patient. Therefore, a scan performed by the AR glasses prior to the wrapping of the device 102 enhances precision of subsequent vein mapping, by first creating a map of the extremity of the body surface.

At step 408, the clinician or the patient applies the wearable device (device 102) by wrapping the wearable device around the extremity of the target site. The wearable device is thus worn by the patient around a limb, the face, or any other part of the body that can be wrapped by the wearable device. The wearable device is flexible and embedded with arrays of sensors (sensors 104) including ultrasound transducers and Doppler sensors that respectively provide sensor data including ultrasound imaging data and blood flow data pertaining the vasculature that lays within the extremity or within the target site.

At step 410, sensor data is acquired by the sensors and wirelessly communicated to the computing unit (unit 108) embedded within the AR glasses (system 110). The sensor data (comprising at least ultrasound data and Doppler data) collected by the sensors is indicative of at least the shape and position of the blood vessels, blood flow within the blood vessels, and shape of the extremity or the outer surface of the body of the patient where the device is worn. In some embodiments, ultrasound data from the ultrasound sensors is in the DICOM format as described above. The Doppler data includes Doppler data that is indicative of blood flow characteristics and is structured in matrices where pixel intensity represents tissue density or flow rate. The computing unit (data processing and analysis unit 108) within the AR glasses is configured to continuously analyze the ultrasound and the Doppler data to: identify optimal veins or arteries for venous or arterial access based on factors such as, but not limited to, vein depth, size, and blood flow velocity; monitor for abnormal blood flow patterns, such as reduced flow indicative of deep vein thrombosis (DVT), venous collapse, or vessel stenosis; provide adaptive real-time feedback, adjusting recommendations based on the patient's dynamic conditions including and not limited to: dehydration, movement, or fluctuations in blood pressure). Some or all of these analyses are performed after merging sensor data from the sensors and optical or visual data from the camera embedded within the AR glasses.

At step 412, the computing unit processes data, as it is configured to do so, by merging the sensor data and the raw optical or visual data from one or more cameras in the AR glasses in order to generate merged data. Raw optical or visual data is collected in real time by the camera(s), such as in step 406, and the analysis is performed in real time.

Therefore, at step 412, the optical or visual data (comprising RGB-D data—which is a combination of standard Red, Green, Blue (RGB) color data with a corresponding Depth (D) data, providing pixel-level distance information (how far away objects are) alongside visual color) from the camera of the AR glasses is sued to provide a 3D surface map or reconstruction of the limb that is covered by the wearable device. The optical or visual data from the AR glasses is spatially aligned with the subsurface vascular structures captured by the ultrasound sensors. The spatial alignment is achieved using coordinate transformation algorithms implemented by the computing unit. Both sources of data—from the sensors of the wearable device and from the camera(s) of the AR glasses, are temporally synced to ensure the vascular structures align with the real-time 3D surface map.

At step 414, the computing unit executes one or more AI algorithms, including machine learning algorithms, to analyze the merged data to generate an accurate vascular map of the patient's extremity. The generated vascular map highlights the veins, arteries and blood flow characteristics (e.g., direction and velocity) of the vasculature within the target site. In some embodiments, the one or more AI algorithms analyze the merged data to accurately identify and highlight (in different colors and/or using textual annotations or labels) one or more candidate veins or arteries suitable for vascular access based on at least vessel depth, vessel diameter, and blood flow characteristics. The wearable device, worn by the patient until now, is removed.

At step 416, the map of the vasculature generated by the computing unit is displayed as a projection onto the patient through the AR glasses. The vascular map is projected onto the patient's extremity, where the wearable device was previously worn. The map of the vasculature is projected as a color-coded 3D visualization of the patient's veins and arteries. In embodiments, different colors in the projection represent different vessel-depths, sizes (in terms of vessel diameters) and blood-flow rates, direction and velocity, for example. In embodiments, the AR projected vascular map is “locked” onto the scanned extremity or area, so that it does not move even when the clinician's head moves.

At step 418, the clinician is aided by the projected display of the vascular map in selecting a suitable venous insertion point to insert a needle for drawing blood, or for treatment, or any other purpose. The AR-enhanced vasculature map projected on the patient extremity enables the clinician to visually inspect and locate the point to access the required vein.

At step 420, the clinician performs the required procedure of inserting the needle. Optionally, AR glasses continue to display the color-coded vasculature map projection during the process of needle insertion.

At step 422, the clinician removes the AR glasses. In some embodiments, patient data collected by the camera(s) and/or the sensors prior to and during the procedure, is securely saved by the computing unit. Optionally, the saved data is securely transmitted to a remote database, such as for example and not limited to a cloud database that may be linked to electronic health record (EHR) of the patient.

At step 424, the saved data is made available for training of AI algorithms (including machine learning algorithms) implemented by the computing unit. The learning and development of AI is useful in future procedures to enable more efficient and consistent vascular access for recurring treatments. Therefore, the AI algorithms of the present specification continually evolve with each procedure.

### Exemplary Use Applications

In embodiments, the system of the present specification provides extensive applications beyond vascular access, catering to both diagnostic and therapeutic needs in various medical disciplines.

### Prenatal Fetal Monitoring

In embodiments, the device of the present specification may also be used for real-time prenatal fetal monitoring. The wearable device can be wrapped around an abdominal area of a pregnant female to track fetal blood flow through the ultrasound transducers and Doppler sensors embedded within the wearable device. Monitoring fetal blood flow is useful in high-risk pregnancies. In addition to the fetal blood flow that flows through an umbilical cord, embodiments of the present specification support monitoring of the placenta and fetal heart rate, while providing real-time feedback to the clinician. The monitoring of the circulation of blood within the placenta enables early detection of placental insufficiency or fetal growth restriction. Continuous monitoring of fetal blood flow patterns is complemented by alerts in cases of detection of abnormal flow velocities. The alerts are generated based on AI computations and are potentially useful in preventing complications during pregnancy. The real-time generation of data and feedback is beneficial to the clinician.

### Vascular Monitoring for Chronic and Post-Surgical Patients

Further, embodiments of the present specification can be used for long-term vascular health monitoring in patients with chronic conditions, such as but not limited to, diabetes, peripheral artery disease (PAD) or venous insufficiency. The combination of the wearable device and the AR system provides for tracking blood flow through critical veins and arteries over time, and for providing early warnings of venous occlusion or thrombus formation. The computing unit embedded within the AR system is configured to save data or wirelessly communicate data to a remote database associated with a specific patient, such as an Electronic Health Record, for tracking the change in sensor data over time. The long-term monitoring that can be provided by the system of the present specification assists clinicians in optimizing treatment strategies and monitoring the effectiveness of therapies.

Additionally, embodiments of the present specification can be used to monitor post-surgery recovery of patients that have undergone vascular surgeries, such as arterial bypass, vein grafts, or stent placement. Continuous post-operative monitoring provided by the embodiments of the present specification enables clinicians to monitor blood flow through the grafts or stented arteries, ensuring patency and detecting early signs of thrombosis or graft failure thereby detecting signs of complications in real-time.

### Pediatric Monitoring

While the embodiments of the present specification have already been described in context of applications in generating vascular maps that aid in accessing veins (for IV catheterization, drawing of blood, among others), these applications are specifically beneficial for pediatric patients. The use of the wearable device and the AR systems reduces the stress associated with repeated insertion of a needle, by increasing the accuracy of needle insertion to the targeted location. Moreover, the present specification reduces risks of complications and increases safety of pediatric patients.

Although the present specification has been described with particular focus on a wearable device for wearing on a limb, the present specification is also designed to be worn on any other anatomical part of the body of a human or an animal. Furthermore, applications of the present specification can extend to various clinical procedures such as and not limited to enabling quick access for fluid resuscitation, blood transfusions, or drug administration, in an emergency trauma care procedure, thereby increasing patient survival rates in critical care scenarios. In emergency settings, particularly in cases of trauma or shock, quick and accurate vascular access is vital. Embodiments of the present specification can also assist with patients undergoing immunotherapy or chemotherapy, where the patients are likely to have fragile veins due to frequent insertions. In such scenarios, infusions are targeted with greater accuracy with the aid of the present specification, which assists in accessing the most suitable veins thereby reducing complications and improving patient comfort. Real time data transmission of ultrasound images and blood flow data enables reviewing and advisory by remotely located physicians.

Therefore, the present specification provides a comprehensive integrated solution for vascular access, blood flow monitoring, and prenatal care. The unique combination of wearable ultrasound and Doppler sensors, AI-driven real-time analysis, and AR-based visualization provides unparalleled accuracy, efficiency, and safety in various medical applications. The present specification addresses critical needs in acute care, chronic disease management, and prenatal health.

The above examples are merely illustrative of the many applications of the systems and methods of present specification. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

What is claimed is:

1. A system for medical imaging, comprising:
a wearable device adapted to be positioned on a patient's target body region, the wearable device comprising:
at least one ultrasound transducer configured to transmit acoustic signals into tissue and receive reflected echoes to generate first data indicative of subsurface vascular structures of the patient's target body region;
at least one Doppler sensor configured to generate second data indicative of blood flow characteristics within blood vessels at the patient's target body region;
a first communication unit in data communication with said at least one ultrasound transducer and said at least one Doppler sensor, wherein the first communication unit is configured to receive the first and second data for onward transmission;
an augmented reality visualization device adapted to be head-worn by a caregiver, the augmented reality visualization device comprising:
a screen configured to present augmented reality content within a field of view of the caregiver when worn;
at least one camera configured to generate third data indicative of a scan of the patient's target body region prior to positioning of the wearable device on the target body region;
a second communication unit configured to receive the first and second data from the first communication unit;
a processor in data communication with said at least one camera, the second communication unit and at least one non-volatile memory for storing a plurality of programmatic instructions, which when executed, cause the processor to:
receive first, second and third data;
process the third data to reconstruct a three-dimensional representation of an anatomical surface of the patient's target body region;
generate fused data by merging the first and third data, wherein the fused data is indicative of a real-time three-dimensional vascular map aligned with the three-dimensional representation of the anatomical surface; and
render the three-dimensional vascular map as an augmented reality overlay that is displayed on the patient's target body region via the display.

2. The system of claim 1, wherein said aligning of the three-dimensional vascular map with the three-dimensional representation of the anatomical surface comprises spatially registering the first data with the third data using one or more coordinate transformation algorithms.

3. The system of claim 2, wherein said aligning of the three-dimensional vascular map with the three-dimensional representation of the anatomical surface further comprises temporally synchronizing the first and third data based on corresponding acquisition timestamps.

4. The system of claim 1, wherein the three-dimensional vascular map is color-coded to represent at least one of vessel depth, vessel diameter, or the blood flow characteristics, and wherein the blood flow characteristics include at least one of blood flow rate, blood flow direction, or blood flow velocity.

5. The system of claim 1, wherein the processor is further configured to execute one or more artificial intelligence algorithms to analyze the first, second and third data to identify and highlight one or more candidate veins or arteries suitable for vascular access based on at least vessel depth, vessel diameter, and blood flow characteristics, and wherein said highlighting is enabled on the three-dimensional vascular map.

6. The system of claim 5, wherein the processor is further configured to store the first, second and third data and to use the stored data as training data for subsequent execution of the one or more artificial intelligence algorithms.

7. The system of claim 1, wherein the augmented reality overlay is spatially locked to the patient's target body region such that the overlay remains aligned with the anatomical surface during movement of the caregiver's head.

8. The system of claim 1, wherein the processor is further configured to analyze the first and second data to monitor vascular health parameters including at least one of blood flow velocity, flow directionality, pulsatility index, resistive index, volumetric flow rate, vessel diameter, vessel cross-sectional area, vessel wall deformation, or changes in vessel compliance over time.

9. The system of claim 8, wherein the processor is further configured to detect vascular abnormalities including at least one of abnormal blood flow, vessel constriction, vessel collapse, or clot formation based on the monitored vascular health parameters.

10. A method for medical imaging, comprising:
positioning a wearable device on a patient's target body region;
generating, using at least one ultrasound transducer, first data indicative of subsurface vascular structures of the patient's target body region;
generating, using at least one Doppler sensor of the wearable device, second data indicative of blood flow characteristics within blood vessels at the patient's target body region;
communicating the first and second data from the wearable device to an augmented reality visualization device worn on a head of a caregiver;
acquiring, using at least one camera of the augmented reality visualization device, third data indicative of a scan of the patient's target body region prior to positioning of the wearable device on the target body region;
processing the third data to reconstruct a three-dimensional representation of an anatomical surface of the patient's target body region;
generating fused data by merging the first data and the third data, wherein the fused data is indicative of a real-time three-dimensional vascular map aligned with the three-dimensional representation of the anatomical surface; and
rendering the three-dimensional vascular map as an augmented reality overlay that is displayed on the patient's target body region via a display of the augmented reality visualization device.

11. The method of claim 10, wherein aligning the three-dimensional vascular map with the three-dimensional representation of the anatomical surface comprises spatially registering the first data with the third data using one or more coordinate transformation algorithms.

12. The method of claim 11, wherein aligning the three-dimensional vascular map with the three-dimensional representation of the anatomical surface further comprises temporally synchronizing the first data and the third data based on corresponding acquisition timestamps.

13. The method of claim 10, wherein rendering the three-dimensional vascular map comprises color-coding the vascular map to represent at least one of vessel depth, vessel diameter, or blood flow characteristics, and wherein the blood flow characteristics include at least one of blood flow rate, blood flow direction, or blood flow velocity.

14. The method of claim 10, further comprising executing one or more artificial intelligence algorithms to analyze the first, second, and third data to identify and highlight one or more candidate veins or arteries suitable for vascular access based on at least vessel depth, vessel diameter, and blood flow characteristics, and wherein said highlighting is enabled on the three-dimensional vascular map.

15. The method of claim 14, further comprising storing the first, second, and third data and using the stored data as training data for subsequent execution of the one or more artificial intelligence algorithms.

16. The method of claim 10, wherein rendering the augmented reality overlay comprises spatially locking the overlay to the patient's target body region such that the overlay remains aligned with the anatomical surface during movement of the caregiver's head.

17. The method of claim 10, further comprising analyzing the first and second data to monitor vascular health parameters including at least one of blood flow velocity, flow directionality, pulsatility index, resistive index, volumetric flow rate, vessel diameter, vessel cross-sectional area, vessel wall deformation, or changes in vessel compliance over time.

18. The method of claim 17, further comprising detecting vascular abnormalities including at least one of abnormal blood flow, vessel constriction, vessel collapse, or clot formation based on the monitored vascular health parameters.
